(19) United States
(12) Patent Application Publication
(10) Pub. No.: US 2014/0094884 A1
(43) Pub. Date: Apr. 3, 2014

(54) WARMING SYSTEM

(71) Applicants: Zaheer Zaidi, Capitola, CA (US); Arezou Zaidi, Capitola, CA (US)

(72) Inventors: Zaheer Zaidi, Capitola, CA (US); Arezou Zaidi, Capitola, CA (US)

(21) Appl. No.: 13/632,799

(22) Filed: Oct. 1, 2012

(51) Int. Cl. A61F 7/08

(52) U.S. Cl. 607/107

(57) ABSTRACT

In an embodiment, a warming system is disclosed. The warming system may include an elastic pad having inner and outer surfaces, wherein a chamber is defined along the inner surface. The warming system may also include a hollow air conveyor positioned within the chamber and supported relative to the elastic pad by the inner surface, wherein the hollow air conveyor has first and second open ends positioned along an air path through the elastic pad, and wherein an inner air vent is defined within the hollow air conveyor and positioned adjacent to a portion of the inner surface such that this portion of the inner surface is exposed to air that is located within the hollow air conveyor.

### TECHNICAL FIELD

The present application relates to the field of warming systems.

### BACKGROUND

The act of warming may involve, for example, increasing the temperature of a particular gas, solid, liquid or plasma. With respect to living organisms, warming processes can be rather important for warm-blooded mammals to avoid sickness, bodily injury and death. This is particularly true of human infants, as they can be relatively sensitive to drops in their respective body temperatures, such as may occur during clothing and/or diaper changing processes. Moreover, applying heat to a strained joint or muscle can oftentimes provide therapeutic relief to an injured individual, as well as aid in the healing of the affected area.

### SUMMARY

This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter.

In an embodiment, a warming system is disclosed. The warming system may include or comprise an elastic pad having inner and outer surfaces, wherein a chamber is defined along the inner surface. The warming system may also include or comprise a hollow air conveyor positioned within the chamber and supported relative to the elastic pad by the inner surface, wherein the hollow air conveyor has first and second open ends positioned along an air path through the elastic pad, and wherein an inner air vent is defined within the hollow air conveyor and positioned adjacent to a portion of the inner surface such that this portion of the inner surface is exposed to air that is located within the hollow air conveyor.

### DESCRIPTION OF THE DRAWINGS

The accompanying drawings, which are incorporated in and form a part of this specification, illustrate embodiments of the present technology, and, together with the Detailed Description, serve to explain principles discussed below.

FIG. 1 is a perspective view of an exemplary warming system in accordance with an embodiment.

FIG. 2 is a cross-sectional plan view of a first exemplary geometric air conveyance arrangement in accordance with an embodiment.

FIG. 3 is a cross-sectional plan view of a second exemplary geometric air conveyance arrangement in accordance with an embodiment.

FIG. 4 is a block diagram of an exemplary heat source configuration in accordance with an embodiment.

FIG. 5 is a perspective view of an exemplary locking arrangement in accordance with an embodiment.

The drawings referred to in this description are not to be understood as being drawn to scale except if specifically noted, and such drawings are only exemplary in nature.

### DETAILED DESCRIPTION

Reference will now be made in detail to embodiments of the present technology, examples of which are illustrated in the accompanying drawings. While the present technology will be described in conjunction with various embodiments, these embodiments are not intended to limit the present technology. Rather, the present technology is to be understood as encompassing various alternatives, modifications and equivalents.

Additionally, in the following Detailed Description, numerous specific details are set forth in order to provide a thorough understanding of the present technology. However, the present technology may be practiced without these specific details. In other instances, well-known methods, procedures, components, and circuits have not been described in detail so as to not unnecessarily obscure aspects of the exemplary embodiments presented herein.

Moreover, when two or more specified devices, apparatuses, components, etc., are herein described as being “coupled with” one another, it is noted that those specified devices, apparatuses, components, etc., are not necessarily attached directly to one another; rather, one or more other devices, apparatuses, components, etc., may be coupled between the two or more specified devices, apparatuses, components, etc.

### Overview

In a first exemplary scenario, a changing pad or mattress is implemented during a clothing and/or diaper changing process. In particular, an infant is positioned on top of the changing pad or mattress, and a diaper (and possibly other clothing) is removed from the infant while the changing pad or mattress supports the infant's body weight. During this process, the infant's body temperature may decrease, thereby causing the infant to become “chilled”, as a result of the removal of the aforementioned clothing, and this chilled state may lead to sickness, bodily injury and even death.

In a second exemplary scenario, a heating pad is implemented to heat the infant during this clothing and/or diaper changing process. In particular, the heating pad houses a series of electrical heating elements configured to give off a degree of heat when powered by electricity. It is noted, however, that the use of such a heating pad may be unsafe. For example, these heating elements may become too hot for an infant's sensitive skin, in which case the infant would most like be unable to effectively communicate (through speech) that the heating pad is causing a burning sensation. Indeed, the heating elements may become damaged (or short-circuited) over time, in which case the probability of these heating elements malfunctioning would increase accordingly.

In contrast to a heating pad that houses a series of electrical heating elements, an embodiment of the present technology provides a warming system that is much safer and significantly more durable. The warming system includes a baby changing pad or mattress and an air conducting system positioned within the body of the pad or mattress. For example, the changing pad or mattress may include a comfortable foam material, in which case the air conducting system may be embedded within the foam of the pad or mattress. The air conducting system, which may be made, for example, of a flame-retardant material (e.g., a flame-retardant plastic material), is sized and positioned such that a degree of even air and/or heat distribution may be achieved within the changing pad or mattress. Moreover, in one embodiment, the pad or mattress is itself constructed of a number of fire-retardant materials so as to further increase a degree of safety associated with the warming system.

Additionally, a heating element is implemented during an operation of the warming system, wherein the heating element is configured to output an amount of heated air. Specifically, the outflow of air from the heating element is injected into the inflow port of the changing pad or mattress, wherein the inflow port leads to the air conducting system positioned within the body of the pad or mattress. The warm air that is circulated through the conducting system warms the material of the pad or mattress, which thereby creates a warm environment for newborns and infants during clothing and/or diaper changing processes. Furthermore, the heating element attaches to a side of the pad or mattress such that the baby's skin never comes into contact with the heating element. Rather the heating element simply injects warm air into the pad or mattress such that the injected air very safely warms the pad or mattress.

In an embodiment, the heating element may be configured to attach to (and detach from) the changing pad or mattress. Indeed, the conducting pad or mattress may include, or be integrated with, a latching mechanism configured to secure the heating element to the pad or mattress. For example, the heating element may be configured to couple with a two-button release mechanism that is integrated with the pad or mattress such that the heating element is secured to the pad or mattress when the heating element is in operation. After this operation has been completed, the two-button release mechanism enables a user to disconnect the heating element from the pad or mattress, thereby promoting easy storage of the various system components. Alternatively, in one embodiment, the detachable unit may itself have a two-button release mechanism configured to separate the heating element from the pad or mattress.

With regard to the detachable heating element, an embodiment provides that the heating element includes a housing, which may be constructed, for example, of a flame-retardant plastic material. The detachable heating element may also include a metal (e.g., aluminum) core, wherein a number of low temperature heating filaments are located inside of the core. Additionally, one or more low flow fans are coupled along an inside, rear portion of the housing so as to be positioned to direct air through, or adjacent to, the heating filament area. When the detachable heating element is attached to the changing pad or mattress, the resulting warm air will flow into the air conducting system of the changing pad or mattress, thereby warming the pad or mattress such as previously discussed.

In view of the foregoing, it is noted that various embodiments of the present technology involve systems and devices that may be utilized for baby changing objectives. It is further noted, however, that the present technology is not limited to such baby changing objectives, and that the present technology may be implemented to achieve a wide range of other objectives.

Various exemplary embodiments of the present technology will now be discussed. It is noted, however, that the present technology is not limited to these exemplary embodiments, and that the present technology also includes obvious variations of the exemplary embodiments and implementations described herein. It is further noted that various well-known components are generally not illustrated in the drawings so as to not unnecessarily obscure various principles discussed herein, but that such well-known components may be implemented by those skilled in the art to practice various embodiments of the present technology.

### Exemplary Systems, Devices, Arrangements and Configurations

Various exemplary systems, devices, arrangements and configurations for implementing various embodiments of the present technology will now be described. However, the present technology is not limited to these exemplary systems, devices, arrangements and configurations. Indeed, other systems, devices, arrangements and configurations may also be implemented.

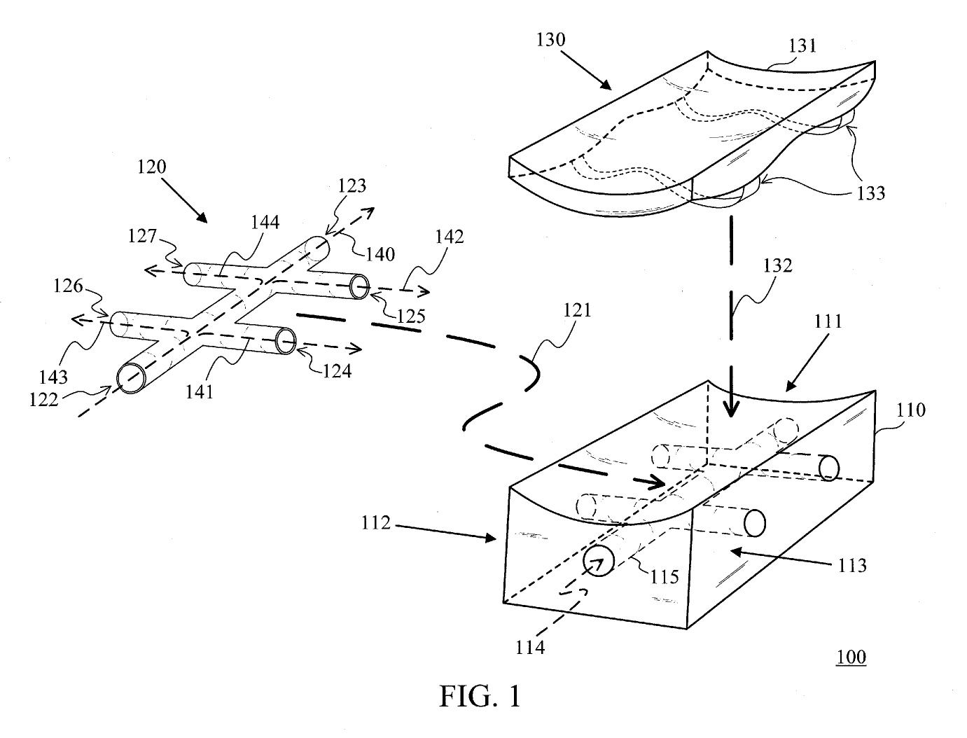

With reference now to FIG. 1, an exemplary warming system 100 in accordance with an embodiment is shown. Warming system 100 includes a pad 110, which, pursuant to one exemplary implementation, may be utilized as a baby changing pad or mattress. Pad 110 includes a material that is capable of absorbing an amount of heat and then radiating this heat from a top surface of pad 110. As such, a baby may be placed on top of pad 110 such that the baby is warmed by the heat radiated by pad 110. In this manner, pad 110 may be implemented to warm an infant during a clothing and/or diaper changing process.

In an embodiment, pad 110 is an elastic pad that may include, for example, a material that is capable of recovering its size and shape subsequent to being deformed. To illustrate, consider the example where pad 110 includes a foam cushion having a predefined size and shape. The foam cushion is capable of deforming in response to a compression force being applied to the foam cushion. The foam cushion is also capable of recovering a predefined size and shape in response to the compression force being removed from the foam cushion.

The foregoing notwithstanding, pursuant to one exemplary implementation, pad 110 includes, or is constructed from, foam or rubber (or combination thereof) material that is relatively firm. As such, when an infant is placed upon pad 110, an elastic nature of the foam/rubber material enables pad 110 to absorb an amount of the infant's weight so as to cushion the infant. However, the foam/rubber material also exhibits a degree of firmness that provides more support to the infant than would a similarly sized air mattress.

In an embodiment, pad 110 includes an open-cell-structured foam material (e.g., foam rubber) that contains a series of interconnected pores. As a result of this interconnected pore network, warm air is able to penetrate into (and pass through) pad 110 relatively easily, as compared to a closed-cell-structured foam material. It is noted, however, that various different materials may be implemented to construct or manufacture pad 110, and that the present technology is not limited to any particular material for pad 110. As such, pad 110 may include a material not discussed herein.

With reference still to FIG. 1, an exemplary embodiment provides that a top, outer surface 111 of pad 110 is curved (or shaped in a concave configuration) such that an object or individual (e.g., a human infant) may be placed on top of pad 110 while the general shape of top, outer surface 111 prevents the object or individual from rolling off of pad 110. Additionally, one or more safety belts may be integrated with pad 110 such that these one or more safety belts may be secured around the infant to help ensure that the infant does not roll off of pad 110.

It is noted, however, that various different geometries for pad 110 may be implemented, and that the present technology is not limited to any particular geometric configuration for pad 110. As such, pad 110 may be sized and shaped in accordance with a geometric configuration not discussed herein. Indeed, and for purposes of illustration, it is noted that top, outer surface 111 of pad 110 may be substantially flat. Moreover, and with reference to the exemplary embodiment shown in FIG. 1, pad 110 is shown as having a number of outer surfaces, such as exemplary outer surfaces 112, 113. Alternatively, pad 110 may be shaped so as to have more or less outer surfaces than are shown in the illustrated embodiment.

In an embodiment, and as previously indicated, pad 110 is capable of absorbing a degree of heat such that a temperature of pad 110 may be increased. In this manner, pad 110 may be heated such that pad 110 will consequently radiate a degree of heat (such as from top, outer surface 111). To illustrate, consider the example where a human infant is placed on top, outer surface 111 of pad 110. Pad 110 is heated such that top, outer surface 111 then transfers a degree of this heat, through a process of conduction, to the human infant. Additionally, pad 110 may also give off a degree of heat into the ambient environment such that the air surrounding the infant is also warm, which in turn has its own, secondary warming affect on the child.

It is noted that various systems, devices and methods may be implemented to heat pad 110, and that the present technology is not limited to any specific system, device or method for heating pad 110. With reference still to FIG. 1, however, an embodiment provides that one or more hollow cavities or chambers, such as exemplary chamber 114, are defined within pad 110. For example, in FIG. 1, chamber is defined within pad 110 along an inner surface 115 of pad 110. As such, and in accordance with one exemplary implementation of exemplary heating system 100, heated air is injected into these one or more hollow cavities or chambers (e.g., into chamber 114) so as to increase a temperature of pad 110.

In addition to the foregoing, and in accordance with an embodiment, exemplary heating system 100 may also include a hollow air conveyor 120. Hollow air conveyor 120, which may include a rigid, fire-retardant material, is positioned within chamber 114, such as indicated by arrow 121, and supported relative to pad 110 by inner surface 115. Hollow air conveyor 120 has first and second open ends 122, 123 positioned along an air path 140 through pad 110. Additionally, and as will be further described herein, one or more inner air vents are defined within hollow air conveyor 120 and respectively positioned adjacent to one or more portions of inner surface 115 such that these one or more portions of inner surface 115 are exposed to air that is located within hollow air conveyor 120. As such, heat may be injected into hollow air conveyor 120 and then routed into pad 110, in a controlled and preselected manner, through these one or more inner air vents.

In an embodiment, hollow air conveyor 120 includes a flame resistant (or fire retardant) material. In this manner, heat may be injected into hollow air conveyor 120 without causing hollow air conveyor 120 to catch on fire. Additionally, hollow air conveyor 120 may include a material that is comparatively more rigid than the material that pad 110 composes, such that a preselected weight (e.g., the weight of an infant) will deform top, outer surface 111 of pad 110 without also deforming hollow air conveyor 120. In this manner, the air way(s) through hollow air conveyor 120 may remain unobstructed. It is noted, however, that the present technology is not limited to any particular material for hollow air conveyor 120, and that various different materials may be implemented to construct hollow air conveyor 120.

With reference still to FIG. 1, it is noted that first and second open ends 122, 123 may define an air intake vent and a first air exhaust vent, respectively, such as where heated air is injected into hollow air conveyor 120 through first open end 122 and then exits hollow air conveyor 120 through second open end 123. In one embodiment, hollow air conveyor 120 may be shaped so as to have a plurality of different exhaust vents. For example, as shown in FIG. 1, hollow air conveyor 120 has third, fourth, fifth and sixth open ends 124-127, which may define second, third, fourth and fifth air exhaust vents, respectively. In this manner, hollow air conveyor 120 is shaped so as to define multiple, distinct air paths 140-144 through pad 110 (when hollow air conveyor 120 is positioned within chamber 114), wherein these multiple, distinct air paths 140-144 are respectively associated with different air exhaust vents.

It is noted that defining additional air paths through hollow air conveyor 120 may have the practical effect of achieving a more even heat distribution throughout pad 110. For example, if a single air exhaust vent is defined within hollow air conveyor 120, then heat will penetrate into pad 110 along the air path (e.g., air path 140) between the air intake vent and the single air exhaust vent. However, if multiple exhaust vents are included, thereby causing a greater number of air paths through hollow air conveyor 120 to be provided, then heat will be distributed into pad 110 along these additional air paths, thereby creating a more even heat distribution.

Furthermore, it is noted that various different geometries for hollow air conveyor 120 may be implemented, and that the present technology is not limited to any particular size and shape for hollow air conveyor 120. However, and with reference to the exemplary embodiment shown in FIG. 1, it is noted that hollow air conveyor 120 may include a number of secondary chambers (e.g., the chambers defined along air paths 141-144) that extend from a primary chamber (e.g., the chamber defined along air path 140), such as in one or more cross configurations (e.g., configurations that are generally “X” or “T” shaped). In this manner, heat may be more evenly distributed to the side or lateral portions of pad 110 rather than simply along the middle or central section of pad 110. This can help to prevent too much heat from being concentrated along a single section of top, outer surface 111 of pad 110, wherein such otherwise high levels of heat may be unsafe for an infant.

With reference still to FIG. 1, and in accordance with an embodiment, exemplary heating system 100 may also include a protective cover 130. Protective cover 130 includes an outer cover 131 sized to conform to an outer surface (e.g., top, outer surface 111) of pad 110, such as indicated by arrow 132. For example, outer cover 131 may include a disposable or washable material (e.g., a washable cloth material) that may be placed on top, outer surface 111 of pad 110 in order to help maintain a level of cleanliness associated with pad 110. To illustrate, an example provides that outer cover 131 is liquid absorbent, such as where bodily fluids from an infant may leak onto outer cover 131 during a clothing and/or diaper changing process. Pursuant to a second example, however, outer cover 131 includes a hydrophobic material, such as to prevent moisture and grime from soaking through outer cover 131 and then coming into contact with pad 110. The foregoing notwithstanding, in a third example, outer cover includes both a layer of liquid absorbent material and a layer of hydrophobic material, such that a degree of moisture may be absorbed into outer cover 131 but then prevented from soaking completely through outer cover 131 so as to come into contact with pad 110.

With reference still to FIG. 1, protective cover 130 may optionally include one or more elastic members, such as exemplary elastic members 133. These one or more elastic members are coupled with outer cover 131 and positioned to engage pad 110 when pad 110 is positioned between at least a portion of outer cover 131 and the one or more elastic members in order to thereby hold outer cover 131 against pad 110. To illustrate, exemplary elastic members 133 are stretched away from outer cover 131 in order to create more space between outer cover 131 and exemplary elastic members 133. Next, pad 110 is repositioned between outer cover 131 and exemplary elastic members 133, and then exemplary elastic members 133 tighten around pad 110 until exemplary elastic members 133 are tight enough to pull outer cover 131 against pad 110 to thereby secure outer cover 131 to pad 110.

As previously indicated, it is noted that various different shapes or geometries for hollow air conveyor 120 may be utilized. For purposes of illustration, a number of exemplary shapes or geometries for hollow air conveyor 120 will now be explored. However, the present technology is not limited to these exemplary shapes or geometries. Indeed, various other shapes or geometries for hollow air conveyor 120 may be implemented.

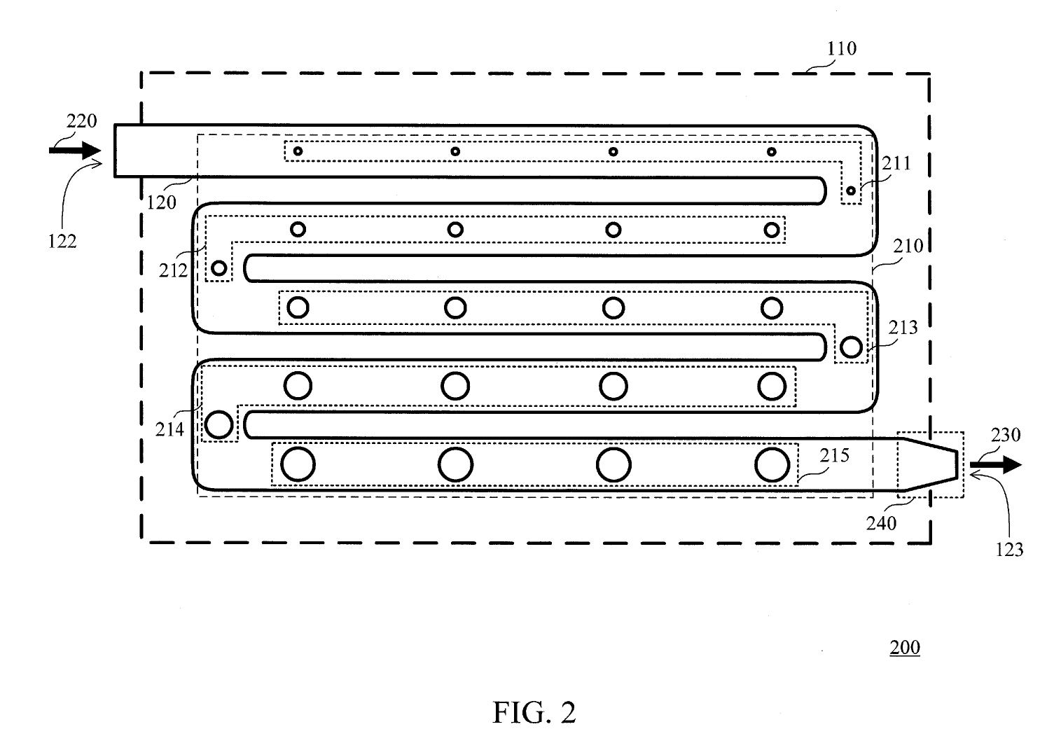

With reference now to FIG. 2, a first exemplary geometric air conveyance arrangement 200 in accordance with an embodiment is shown. In particular, hollow air conveyor 120, which may include a flame resistant (or fire retardant) material, is positioned within pad 110 (such as in chamber 114 shown in FIG. 1). A plurality of inner air vents 210 are defined within hollow air conveyor 120 and respectively positioned adjacent to portions of inner surface 115 of pad 110 (see, e.g., FIG. 1) such that these portions of inner surface 115 are exposed to air that is located within hollow air conveyor 120. As such, heat may be injected into hollow air conveyor 120 and then routed into pad 110, in a controlled and preselected manner, through these inner air vents 210.

In an embodiment, chamber 114 of pad 110 completely extends through pad 110 in a straight or linear fashion. In this manner, air may pass through pad 110 along a single vector direction. In accordance with one embodiment, however, and with reference to the embodiment shown in FIG. 2, chamber 114 (and similarly hollow air conveyor 120) twists, bends or curves through pad 110 such that a surface area of inner surface 115 is increased (see also, e.g., FIG. 1). Consequently, the number of inner air vents 210 that may be implemented also increases, and the heat transferred to pad 110 through these inner air vents 210 is more evenly distributed throughout pad 110 rather than being localized along a single, linear portion of pad 110.

Pursuant to an embodiment, inner air vents 210 are respectively positioned within hollow air conveyor 120 based upon a heat distribution associated with pad 110 as a function of a preselected temperature range of the injected air. To illustrate, consider the example where pad 110 is to be utilized as a clothing/diaper changing station, wherein an infant is to be warmed by pad 110 during a clothing and/or diaper changing process in order to prevent the infant from becoming ill. A temperature of pad 110 that is to be achieved with respect to this objective is identified, and a temperature range for the air that is to be injected into hollow air conveyor 120, such that the aforementioned temperature of pad 110 may be achieved, is then determined, wherein the heat distribution associated with pad 110 is a function of the temperature of the injected air. The respective positions of inner air vents 210 are then selected based upon this heat distribution, such that a relatively even heat distribution is achieved along top, outer surface 111 (see, e.g., FIG. 1) of pad 110.

To further illustrate, an embodiment provides that an amount of air, which has been heated to a first temperature, is injected into first open end 122 of hollow air conveyor 120, as indicated by injection vector 220. This heated air travels through a first portion of hollow air conveyor 120 such that portions of this air travel through a first set of inner air vents 211, thereby warming the adjacent portions of inner surface 115 (see, e.g., FIG. 1) of pad 110, and such that another portion of this heated air (e.g., the residual air) continues to travel through hollow air conveyor 120. The residual air then travels through a second portion of hollow air conveyor 120 such that portions of this residual air travel through a second set of inner air vents 212. However, in one exemplary implementation, the injected air begins to cool as it travels through hollow air conveyor 120, thereby causing the temperature of the portions of the residual air traveling through second set of inner air vents 212 to be lower than the temperature of the portions of the heated air traveling through first set of inner air vents 212. As such, the distances between neighboring or adjacent air vents (or between their respective centers) from among second set of inner air vents 212 may be selected to be relatively lower than the distances between neighboring or adjacent air vents (or between their respective centers) from among first set of inner air vents 211, thereby causing a relatively denser air vent distribution to be associated with the second portion of hollow air conveyor 120 than is associated with the first portion of hollow air conveyor 120. Consequently, a greater amount of surface area of inner surface 115 will be exposed to the portions of residual air in the aforementioned second portion of hollow air conveyor 120 than will be exposed to the portions of heated air in the aforementioned first portion of hollow air conveyor 120. In this manner, the distances between neighboring or adjacent inner air vents (or between their respective centers) may be selected such that a more uniform heat distribution occurs through pad 110.

The foregoing notwithstanding, in an embodiment, first and second open ends 122, 123 of hollow air conveyor 120 define an air intake vent and an air exhaust vent, respectively. The air path through hollow air conveyor 120 begins at the air intake vent, as indicated by injection vector 220, and ends at the air exhaust vent, as indicated by exhaust vector 230. Additionally, inner air vents 210 each have a different width or diameter, and inner air vents 210 are arranged within hollow air conveyor 120 such that an increasing air vent width or diameter sequence is defined along the air path. Consequently, a greater amount of surface area of inner surface 115 will be exposed to the portions of residual air in the aforementioned second portion of hollow air conveyor 120 than will be exposed to the portions of heated air in the aforementioned first portion of hollow air conveyor 120. Thus, increasing the width or diameter of subsequent air vents along the air path of the injected air helps to counteract the natural cooling effect that the injected air is subjected to along this air path by decreasing the resistance to air flow exhibited by the subsequent inner air vents. In this manner, the increasing air vent width or diameter sequence may be selected such that a more uniform heat distribution occurs through pad 110.

Moreover, in one embodiment, each of the different widths or diameters of inner air vents 210 are selected based upon a heat distribution associated with the pad 110 as a function of a preselected temperature range of the injected air. To illustrate, consider the example where pad 110 is to be utilized as a clothing/diaper changing station, wherein an infant is to be warmed by pad 110 during the clothing and/or diaper changing process in order to prevent the infant from becoming ill. A temperature of pad 110 that is to be achieved with respect to this objective is identified, and a temperature range for the air that is to be injected into hollow air conveyor 120 such that the aforementioned temperature of pad 110 may be achieved is then determined, wherein the heat distribution associated with pad 110 is a function of the temperature of the injected air. The widths or diameters of inner air vents 210 are then selected based upon this heat distribution, such that a relatively even heat distribution is achieved along top, outer surface 111 of pad 110.

To further illustrate, an embodiment provides that an amount of air, which has been heated to a first temperature, is injected into first open end 122 of hollow air conveyor 120, as indicated by injection vector 220. This heated air travels through the first portion of hollow air conveyor 120 such that portions of this air travel through first set of inner air vents 211, thereby warming the adjacent portions of inner surface 115 of pad 110. The residual air then travels through a second portion of hollow air conveyor 120 such that portions of this residual air travel through second set of inner air vents 212. However, as previously noted, an exemplary implementation provides that the injected air begins to cool as it travels through hollow air conveyor 120, thereby causing the temperature of the portions of the residual air traveling through second set of inner air vents 212 to be lower than the temperature of the portions of the heated air traveling through first set of inner air vents 212. As such, the widths or diameters of the air vents from among second set of inner air vents 212 may be selected to be relative larger than the widths or diameters of the air vents from among first set of inner air vents 211. Consequently, a greater amount of surface area of inner surface 115 will be exposed to the portions of residual air in the aforementioned second portion of hollow air conveyor 120 than will be exposed to the portions of heated air in the aforementioned first portion of hollow air conveyor 120.

With reference still to FIG. 2, and in accordance with an embodiment, the injected air will travel through portions of hollow air conveyor 120 associated with first, second, third, fourth and fifth sets of inner air vents 211-215, respectively (and in that order). As such, it is noted that: (1) the widths or diameters of the inner air vents from among second set of inner air vents 212 are longer than the widths or diameters of the inner air vents from among first set of inner air vents 211, (2) the widths or diameters of the inner air vents from among third set of inner air vents 213 are longer than the widths or diameters of the inner air vents from among second set of inner air vents 212, (3) the widths or diameters of the inner air vents from among fourth set of inner air vents 214 are longer than the widths or diameters of the inner air vents from among third set of inner air vents 213, and (4) the widths or diameters of the inner air vents from among fifth set of inner air vents 215 are longer than the widths or diameters of the inner air vents from among fourth set of inner air vents 214. Moreover, it is noted that the widths or diameters of the inner air vents within each set of inner air vents may also increase along the aforementioned air path through hollow air conveyor 120.

Furthermore, in an embodiment, one or more ends of hollow air conveyor 120 that function as exhaust ports (e.g., second open end 123 shown in FIG. 2) have a tapered geometry, such as the exemplary tapered geometry 240 shown in FIG. 2. In particular, these one or more exhaust ports are tapered so as to slow the escape of the heated air from hollow air conveyor 120. In this manner, the heated air is trapped in hollow air conveyor 120 for a longer period of time, which enables heat to continue to build within hollow air conveyor 120, thereby causing more heat to travel through inner air vents 210 (and thereby more efficiently and effectively warming inner surface 115 of pad 110). Indeed, in one embodiment, the respective widths or diameters of these exhaust ports are selected based upon a heat distribution associated with the pad 110 as a function of a preselected temperature range of the injected air.

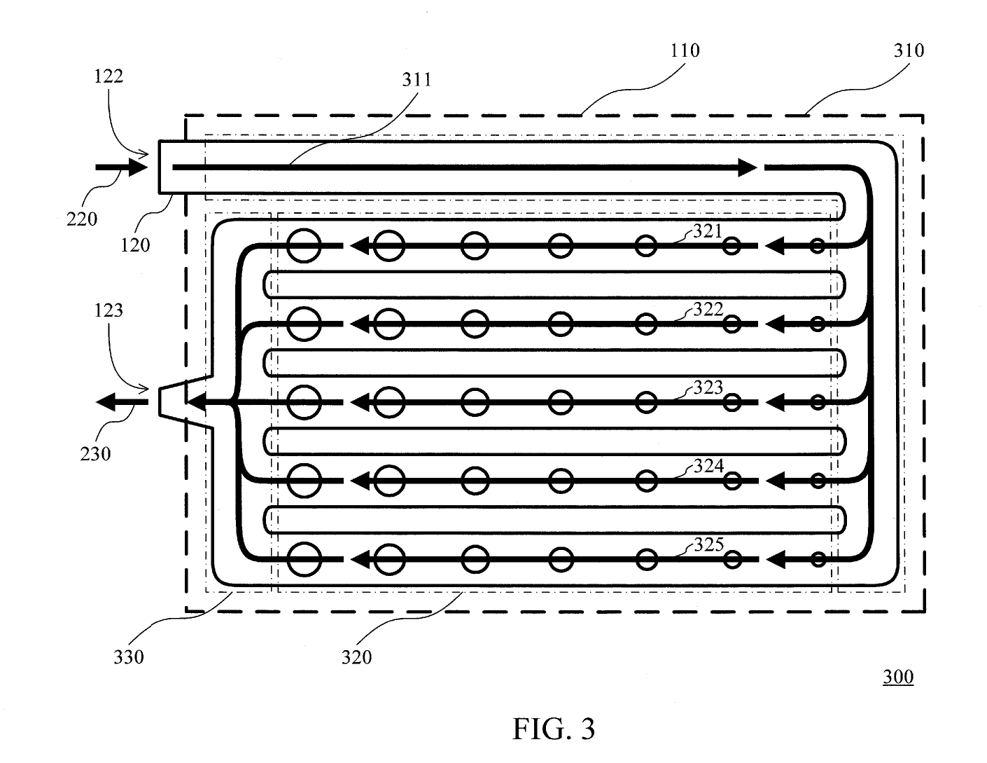

With reference now to FIG. 3, a second exemplary geometric air conveyance arrangement 300 in accordance with an embodiment is shown. In particular, hollow air conveyor 120, which may include a flame resistant (or fire retardant) material, is positioned within pad 110 (such as in chamber 114 shown in FIG. 1). Additionally, a number of different air chambers are defined within hollow air conveyor 120 such that a plurality of different air paths are defined through pad 110. It is noted that providing a plurality of different air paths through which the heated air may travel through pad 110 may increase a degree of uniformity associated with the heat distribution through pad 110.

To illustrate, consider the example where heated air is injected into hollow air conveyor 120 through first open end 122, as indicated by injection vector 220. This heated air initially travels through a primary air chamber 310, as indicated by air path 311. After this primary air chamber 310, hollow air conveyor 120 is divided into a plurality of different air chambers 320, and the injected air then travels through these different air chambers 320, as indicated by air paths 321-325. In particular, the injected air is divided into different portions based upon the number of possible paths, and each of these portions of the divided air then travel through one of these different air chambers 320.

In an embodiment, inner air vents are positioned along these different air chambers 320 such that the heated air within these chambers is able to heat inner surface 115 (see, e.g., FIG. 1) of pad 110, such as previously described herein. It is noted that the density, and/or selected length of the respective widths or diameters, of these inner air vents may increase along the respective air paths, such as previously described herein. To illustrate, an example provides that the distances between neighboring or adjacent air vents (or between their respective centers) in the chamber associated with air path 321 decreases along air path 321, such that a density of these air vents increases along air path 321. Moreover, in a second example, the selected length of the respective widths or diameters of these air vents increases along air path 321, such that a greater amount of surface area of inner surface 115 is exposed to the heated air as this heated air travels along air path 321.

With reference still to FIG. 3, it is noted that the divided portions of the injected air finally converge in a converging air chamber 330, and the converged air then exits hollow air conveyor 120 through second open end 123, as indicated by exhaust vector 230. Furthermore, in an embodiment, one or more ends of hollow air conveyor 120 that function as exhaust ports (e.g., second open end 123 shown in FIG. 3) have a tapered geometry. As previously noted, these one or more exhaust ports are tapered so as to slow the escape of the heated air from hollow air conveyor 120. In this manner, the heated air is trapped in hollow air conveyor 120 for a longer period of time, which enables heat to continue to build within hollow air conveyor 120, thereby causing more heat to travel through inner air vents 210 (and thereby more efficiently and effectively warming inner surface 115 of pad 110). Indeed, in one embodiment, the respective widths or diameters of these exhaust ports are selected based upon a heat distribution associated with the pad 110 as a function of a preselected temperature range of the injected air.

With reference again to FIG. 1, in an embodiment, first and second open ends 122, 123 of hollow air conveyor 120 define an air intake vent and an air exhaust vent, respectively. Additionally, and as previously indicated, warming system 100 may further include a heat source configured to inject an amount of heated air into the air intake vent such that a first portion of the heated air escapes the hollow air conveyor 120 through a plurality of inner air vents defined in hollow air conveyor 120 so as to warm the portions of inner surface 115 that are respectively positioned adjacent to these inner air vents, and such that a second portion of the heated air escapes the hollow air conveyor 120 through the air exhaust vent. In view of the foregoing, an exemplary configuration for such a heat source will now be explored. It is noted, however, that the present technology is not limited to this exemplary configuration, and that various other configurations for such a heat source may be implemented.

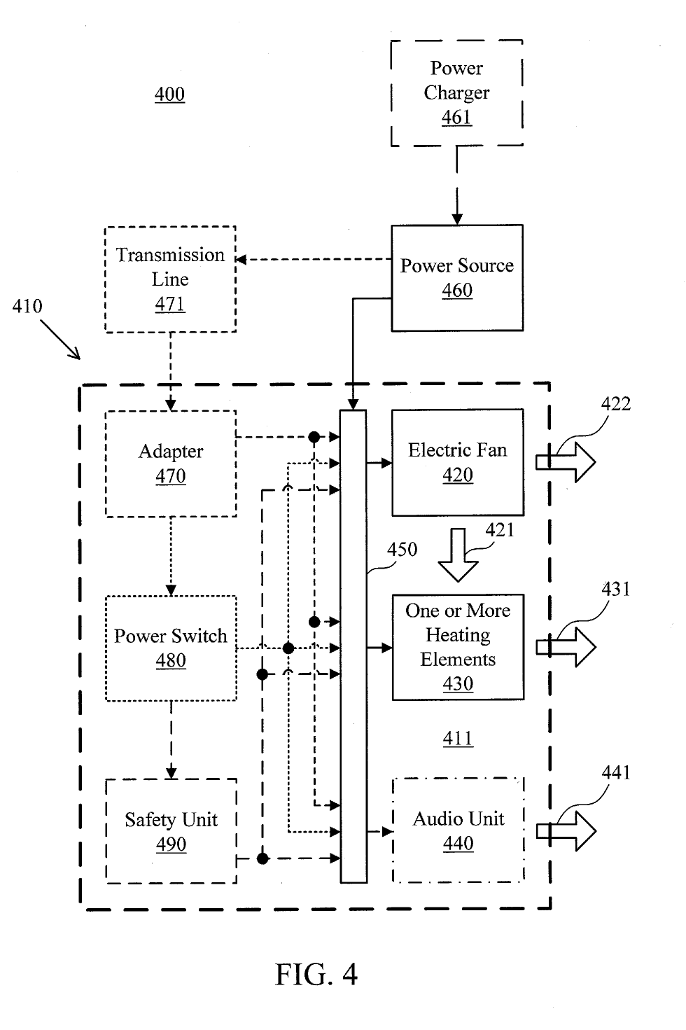

With reference now to FIG. 4, an exemplary heat source configuration 400 in accordance with an embodiment is shown. In particular, a heat source 410 includes a base member 411 (e.g., a housing). Heat source 410 also includes an electric fan 420 associated or coupled with base member 411, wherein electric fan 420 is configured to generate an initial air current 421 when electric fan 420 is powered by a preselected amount of electrical current. Heat source 410 also includes one or more heating elements 430 associated or coupled with base member 411, wherein one or more heating elements 430 are configured to generate a degree of heat when powered by a preselected amount of electrical current. Moreover, when initial air current 421 passes adjacent to one or more heating elements 430, this initial air current 421 is warmed such that a warm air current 431 is generated. In this manner, heat source 410 is able to generate a degree of heated air, which may then be injected into hollow air conveyor 120.

In an exemplary implementation, and with reference to FIGS. 1 and 4, heat source 410 is implemented to inject heated air into hollow air conveyor 120 so as to warm pad 110. Additionally, an infant is placed on top of pad 110, and a sound 422 emitted by electric fan 420 during an operation of electric fan 420 serves to calm the infant during the clothing and/or diaper changing processes. Indeed, in one embodiment, electric fan 420 includes one or more fan blades, and the geometry of these one or more fan blades is chosen such that these one or more fan blades will emit, during an operation of electric fan 420, a sound 422 having a preselected frequency (e.g., a sound that is soothing to an infant).

The foregoing notwithstanding, in an embodiment, heat source optionally includes an audio unit 440 associated or coupled with base member 411. Audio unit 440 is configured to output a preselected audio signal 441 when audio unit 440 is powered by a preselected degree of electrical current. To illustrate, consider the example where pad 110 of warming system 100 is to be implemented as a clothing/diaper changing station. Heat source 410 is used to inject heat into hollow air conveyor 120 such that pad 110 is heated during a clothing and/or diaper changing process. Additionally, audio unit 440 of heat source 410 outputs preselected audio signal 441, which may include, for example, a series of audible sounds (e.g., a song) that is soothing to the infant. In this manner, a baby (e.g., an infant that is suffering from colic) can be calmed during the clothing and/or diaper changing process such that this process can be accomplished more easily, quickly and efficiently.

Thus, an embodiment provides that each of electric fan 420 and one or more heating elements 430 (and optionally audio unit 440) are provided with a degree of electric power such that their respective operations may be carried out. In one embodiment, heat source 410 includes one or more electrically conductive members 450 (e.g., one or more metal wires) associated or coupled with electric fan 420 and one or more heating elements 430 (and optionally with audio unit 440). Additionally, a power source 460, which is associated or coupled with one or more electrically conductive members 450, is configured to provide a degree of electric power to one or more electrically conductive members 450 such that one or more electrically conductive members 450 are able to transmit various amounts of this power to electric fan 420 and one or more heating elements 430 (and optionally audio unit 440), respectively.

In an embodiment, power source 460 is a local power source that is attached to or housed within base member 411. For example, a battery compartment may be defined within base member 411, and power source 460 may include one or more batteries (or electrochemical cells) that are respectively positioned within this battery compartment such that these one or more batteries are able to transmit electric power to one or more electrically conductive members 450. Additionally, an embodiment provides that power source 460 is capable of being electrically recharged (e.g., by an optional power charger 461). Pursuant to one embodiment, however, power source 460 is an external power source (e.g., an alternating current (AC) power source associated with a power socket that is embedded within a wall of a building). Thus, it is noted that the present technology is not limited to any particular type of electric power source.

In accordance with an exemplary implementation, power source 460 is coupled with one or more electrically conductive members 450 such that power source 460 is capable of providing the aforementioned electric power directly to at least one electrically conductive member from among one or more electrically conductive members 450. However, in one embodiment, such as where power source 460 is an external power source, heat source 410 includes an adapter 470 that is coupled or associated with base member 411. Additionally, a transmission line 471 is associated or coupled with adapter 470, wherein transmission line 471 is also sized and configured to be coupled with or attached to power source 460 (e.g., by electrically connecting transmission line 471 to power source 460) during an operation of heat source 410. Transmission line 471 includes an electrically conductive material such that transmission line 471 is able to transmit electric power from power source 460 to adapter 470 subsequent to transmission line 471 being coupled with or attached to power source 460. In this manner, external power may be routed to heat source 410.

Furthermore, in an embodiment, adapter 470 includes a mechanical, or electro-mechanical, adapter that is configured to receive a degree of direct current (DC) from transmission line 471. For example, adapter 470 may include a universal serial bus (USB) connector, and transmission line 471 may be a USB cable that is sized to plug into, or attach to, the USB connector so as to electrically couple or connect adapter 470 and transmission line 471. Indeed, in one embodiment, transmission line 471 is configured to plug into a cigarette lighter receptacle in an automobile, wherein the cigarette lighter receptacle functions as a DC connector that functions to supply electrical power for portable accessories used in or near the automobile. In this manner, heat source 410 may be used in conjunction with warming system 100 (shown in FIG. 1) by traveling parents to change their infant when they are away from home.

The foregoing notwithstanding, an embodiment provides that adapter 470 includes a power converter, such as a DC to DC power converter that is configured to reduce the amount of direct current received from transmission line 471, or such as an AC to DC power converter that is configured to convert a degree of alternating current received from transmission line 471 into direct current that may be used to power heat source 410. It is noted, however, that the present technology is not limited to the implementation of any particular type of power converter. Indeed, various other power converters not discussed herein may also be implemented.

In an embodiment, adapter 470 is associated or coupled with one or more electrically conductive members 450 such that adapter 470 is able to provide the received (or converted) power directly to one or more electrically conductive members 450. Pursuant to one embodiment, however, heat source 410 optionally includes a power switch 480, wherein power switch 480 is electrically associated or coupled with adapter 470 such that power is routed from adapter 470 to one or more electrically conductive members 450 when power switch 480 is “closed” (e.g., when the electrical contacts of power switch 480 are physically touching one another such that electricity is able to flow between them), but not when power switch 480 is “open” (e.g., when the electrical contacts of power switch 480 are not physically touching one another such that electricity is unable to flow between them). In this manner, a user of heat source 410 may utilize power switch 480 to both initiate (e.g., turn “on”) and terminate (e.g., turn “off”) an operation of the various components of heat source 410 (e.g., electric fan 420, one or more heating elements 430 and audio unit 440) simply by toggling power switch 480 to its “closed” and “opened” states, respectively.

Thus, in accordance with an embodiment, power switch 480 is configured to enable a user to initiate (or “POWER ON”) an operation of heat source 410 as well as terminate (or “POWER OFF” an operation of heat source 410. Alternatively, or in addition to the forgoing, an embodiment provides that power switch 480 enables a user to switch between a low power setting and a high power setting (and possibly one or more medium settings). For example, a selection of a higher power setting would cause electric fan 420 to turn relatively faster and/or cause one or more heating elements 430 to be heated to a relatively higher temperature. In this manner, the heat emitted by heat source 410 could be controlled by the user based on the available settings provided by power switch 480. Consequently, the user would have the option of warming pad 110 to, for example, a comfortable, mid-range temperature (rather than to the maximum available temperature).

The foregoing notwithstanding, an embodiment provides that heat source 410 optionally includes a safety unit 490 configured to prevent relatively high degrees of electrical current from damaging the components of power source 460. For example, safety unit 490 may be associated or coupled with both power switch 480 and one or more electrically conductive members 450 such that electrical current having a signal strength or amplitude equal to or below a predefined threshold is able to flow from power switch 480, and through safety unit 490, in order to reach one or more electrically conductive members 450. However, if the signal strength or amplitude of this electrical current is above this predefined threshold, then safety unit 490 will prevent this current from reaching one or more electrically conductive members 450. In accordance with an exemplary implementation, safety unit 490 includes an electrical fuse and/or circuit breaker. It is noted, however, that the present technology is not limited to any particular type of safety unit. Indeed, other types of safety units not mentioned herein may be implemented.

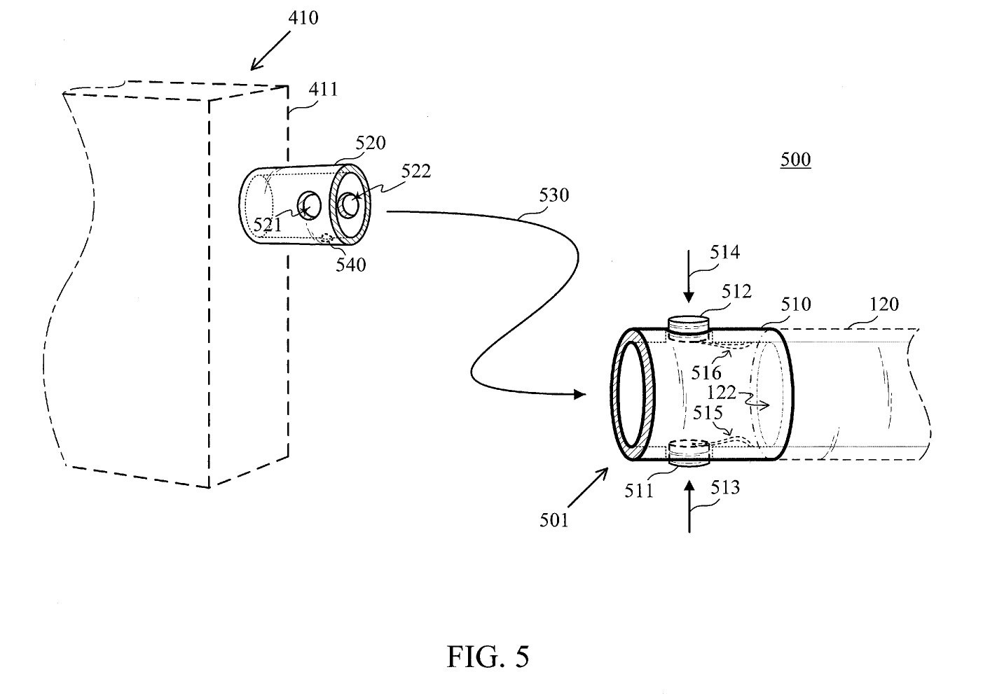

With reference now to FIG. 5, an exemplary locking arrangement 500 in accordance with an embodiment is shown. In particular, a locking mechanism 501 is provided, wherein locking mechanism 501 is configured to couple or connect heat source 410 with hollow air conveyor 120 such that heat source 410 is positioned to inject warm air into hollow air conveyor 120.

To illustrate, and with reference again to FIG. 1, an embodiment provides that first and second open ends 122, 123 of hollow air conveyor 120 define an air intake vent and a first air exhaust vent, respectively. Also, and with reference to FIGS. 4 and 5, heat source 410 includes an exhaust port 520 that is coupled with, or extends from, base member 411, wherein electric fan 420 is positioned to blow initial air current 421 such that warm air current 431 escapes base member 411 through exhaust port 520. Additionally, first and second locking receptacles 521, 522 are associated with heat source 410, such as where first and second locking receptacles 521, 522 are defined within exhaust port 520. Moreover, locking mechanism 501, which includes a base 510 that is coupled with, or extends from, hollow air conveyor 120, has first and second locking extensions 511, 512, wherein first and second locking extensions 511, 512 are configured to extend from base 510 to first or second extended positions, respectively (such as shown in FIG. 5), as well as retract toward (or into) base 510 to first and second retracted positions, respectively. Furthermore, first and second locking extensions 511, 512 are sized to engage first and second locking receptacles 521, 522, respectively, when first and second locking extensions 511, 512 are in the first and second extended positions, respectively (such as shown in FIG. 5), so as to couple or connect heat source 410 with the air intake vent of hollow air conveyor 120 such that heat source 410 is positioned to inject heated air into the air intake vent.

To further illustrate, consider the example where at least a portion of base 510 is sized to fit within exhaust port 520 (as indicated by arrow 530) when first and second locking extensions 511, 512 are pushed or retracted toward base 510 in first and second retraction directions 513, 514, respectively. Once base 510 is repositioned inside of exhaust port 520 such that first and second locking extensions 511, 512 are aligned adjacent to first and second locking receptacles 521, 522, respectively, first and second locking extensions 511, 512 automatically spring away from base 510 such that first and second locking extensions 511, 512 are repositioned within first and second locking receptacles 521, 522, respectively. It is noted that this springing action may result, for example, from first and second spring members 515, 516 exerting mechanical forces on first and second locking extensions 511, 512, respectively, wherein first and second spring members 515, 516 couple or connect first and second locking extensions 511, 512 with locking mechanism 501. As a result, exhaust port 520, and consequently heat source 410, is attached to hollow air conveyor 120. In particular, exhaust port 520 is aligned with first open end 122 of hollow air conveyor 120 such that heat source 410 is positioned to inject heated air through the air intake vent of hollow air conveyor 120.

Thus, an exemplary mechanical configuration provides that base 510 of locking mechanism 501 extends from, or is integrated with, hollow air conveyor 120, and that first and second locking receptacles 521, 522 are defined within exhaust port 520 of heat source 410. It is noted, however, that the present technology is not limited to this exemplary mechanical configuration. Indeed, various other mechanical configurations not discussed herein may also be implemented.

For example, one embodiment provides that base 510 of locking mechanism 501 extends from, or is integrated with, exhaust port 520 of heat source 410. Additionally, first and second locking receptacles 521, 522 are defined within a portion of hollow air conveyor 120 that is near or adjacent to first open end 122. Consequently, base 510 may be positioned inside of first open end 122 of hollow air conveyor 120, such that first and second locking extensions 511, 512 are aligned adjacent to first and second locking receptacles 521, 522, respectively. Once first and second locking extensions 511, 512 engage first and second locking receptacles 521, 522, respectively, such as previously discussed, then exhaust port 520, and consequently heat source 410, will be attached to hollow air conveyor 120 such that heat source 410 is positioned to inject heated air through the air intake vent of hollow air conveyor 120.

In addition to the foregoing, it is also noted that a safety unit (e.g., safety unit 490) may be implemented to prevent heated air from being actively dispersed from heat source 410 in the event that heat source 410 becomes suddenly dislodged from hollow air conveyor 120. To illustrate, consider the example where safety unit 490 is electrically coupled with a safety tab or switch 540, wherein safety tab or switch 540 may be positioned, for example, in exhaust port 520 (such as shown in FIG. 5) such that a mechanical force is exerted on safety tab or switch 540 by base 510 when base 510 is inserted into exhaust port 520. This mechanical force causes safety tab or switch 540 to close a safety circuit with which safety unit 490 is electronically coupled, such that a safety signal is sent to safety unit 490. In response to receiving this safety signal, safety unit 490 will enable an operation of electric fan 420 and one or more heating elements 430. However, if base 510 is pulled out of exhaust port 520, such that this mechanical force is no longer exerted on safety tab or switch 540, then safety tab or switch 540 opens the aforementioned safety circuit, which ceases the aforementioned safety signal, thereby causing safety unit 490 to cease the operation of electric fan 420 and one or more heating elements 430. In this manner, the connection of exhaust port 520 to hollow air conveyor 120 may be condition precedent to heat source 410 blowing warm air through exhaust port 520. Consequently, the accidental blowing of hot air directly onto an individual's skin during an implementation of heat source 410 may be avoided.

Thus, an embodiment provides that heat source 410 is configured to inject warm air into hollow air conveyor 120. It is noted that the term “air” as used herein may be defined as referring to any gaseous substance, which may or may not be a mixture of different types of gases. Indeed, the present technology is not limited to any particular type of gaseous substance.

It is further noted that the present technology is also not limited to the use of a heated gaseous substance for warming pad 110. Indeed, one embodiment provides that a substance other than a gaseous substance (e.g., a heated liquid) is pumped into the hollow conveyor of pad 110 (in which case hollow air conveyor 120 may be simply referred to as a “conveyor”). For example, a heat source may be implemented that is capable of heating a liquid (e.g., water) and then injecting the heated liquid into the conveyor of pad 110. Additionally, a hose (e.g., a plastic or rubber hose) may be implemented to circulate the liquid back to the heat source, such as where the liquid that escapes through the second open end 123 is routed back to the heat source, whereby the liquid will be reheated and then re-injected into the conveyor of pad 110.

Finally, it is noted that heat source 410 may be configured to coupled directly with hollow air conveyor 120 (e.g., by attaching to locking mechanism 501), such as in a manner described herein. Pursuant to one embodiment, however, a hose (e.g., a plastic or rubber hose) is coupled between exhaust port 520 and hollow air conveyor 120 such that this hose is able to direct the heated air from heat source 410 into first open end 122 of hollow air conveyor 120. In this manner, heat source 410 may be positioned a farther distance away from pad 110, which could potentially offer a number of additional safety benefits.

### Summary Concepts

It is noted that the foregoing discussion has presented at least the following concepts:

Concept 1. A warming system including or comprising:

an elastic pad having inner and outer surfaces, a chamber being defined along the inner surface; and

a hollow air conveyor positioned within the chamber and supported relative to the elastic pad by the inner surface, the hollow air conveyor having first and second open ends positioned along an air path through the elastic pad, and an inner air vent being defined within the hollow air conveyor and positioned adjacent to a portion of the inner surface such that the portion of the inner surface is exposed to air that is located within the hollow air conveyor.

Concept 2. The warming system of Concept 1, further including or comprising:

a protective cover including or comprising:
an outer cover sized to conform to the outer surface; and
an elastic member coupled with the outer cover and positioned to engage the elastic pad when the elastic pad is positioned between at least a portion of the outer cover and the elastic member to thereby hold the outer cover against the elastic pad.
   Concept 3. The warming system of Concept 1, wherein the first and second open ends define an air intake vent and a first air exhaust vent, respectively, the hollow air conveyor having a third open end that defines a second air exhaust vent such that the hollow air conveyor is shaped so as to define multiple air paths through the elastic pad that are respectively associated with different air exhaust vents.
   Concept 4. The warming system of Concept 1, wherein a plurality of different air chambers are defined within the hollow air conveyor such that a plurality of different air paths are defined through the elastic pad.
   Concept 5. The warming system of Concept 1, wherein the hollow air conveyor includes or comprises a flame resistant material, a plurality of inner air vents being defined within the flame resistant material and respectively positioned adjacent to portions of the inner surface such that the portions of the inner surface are exposed to air that is located within the hollow air conveyor.
   Concept 6. The warming system of Concept 5, wherein the plurality of inner air vents are respectively positioned within the flame resistant material based upon a heat distribution associated with the elastic pad as a function of a preselected temperature range of the air.
   Concept 7. The warming system of Concept 5, wherein the first and second open ends define an air intake vent and an air exhaust vent, respectively, the air path beginning at the air intake vent and ending at the air exhaust vent, the plurality of inner air vents each having a different width or diameter, and the plurality of inner air vents being arranged within the flame resistant material such that an increasing air vent width or diameter sequence is defined along the air path.
   Concept 8. The warming system of Concept 7, wherein each of the different widths or diameters are selected based upon a heat distribution associated with the elastic pad as a function of a preselected temperature range of the air.
   Concept 9. The warming system of Concept 5, wherein the first and second open ends define an air intake vent and an air exhaust vent, respectively, the warming system further including or comprising:

a heat source configured to inject an amount of heated air into the air intake vent such that a first portion of the heated air escapes the hollow air conveyor through the plurality of inner air vents so as to warm the portions of the inner surface, and such that a second portion of the heated air escapes the hollow air conveyor through the air exhaust vent.

Concept 10. The warming system of Concept 9, wherein first and second locking receptacles are associated with the heat source, the warming system further including or comprising:

a locking mechanism including or comprising:
a base; and
first and second locking extensions each configured to extend from the base to first or second extended positions, respectively, or retract toward the base to first and second retracted positions, respectively, the first and second locking extensions sized to engage the first and second locking receptacles, respectively, when the first and second locking extensions are in the first and second extended positions, respectively, so as to couple the heat source with the air intake vent such that the heat source is positioned to inject the heated air into the air intake vent.
   Concept 11. A warming system including or comprising:

a pad having a chamber defined therein such that the chamber defines an air path through the pad.

Concept 12. The warming system of Concept 11, further including or comprising:

a heat source configured to coupled with the pad and inject an amount of heated air into the chamber.

Although various exemplary embodiments of the present technology are described herein in a language specific to structural features and/or methodological acts, the subject matter defined in the appended claims is not necessarily limited to the specific features or acts described above. Rather, the specific features and acts described above are disclosed as exemplary forms of implementing the claims.

What is claimed is:

1. A warming system comprising:
an elastic pad having inner and outer surfaces, a chamber being defined along said inner surface; and
a hollow air conveyor positioned within said chamber and supported relative to said elastic pad by said inner surface, said hollow air conveyor having first and second open ends positioned along an air path through said elastic pad, and an inner air vent being defined within said hollow air conveyor and positioned adjacent to a portion of said inner surface such that said portion of said inner surface is exposed to air that is located within said hollow air conveyor.

2. The warming system of claim 1, further comprising:
a protective cover comprising:
an outer cover sized to conform to said outer surface; and
an elastic member coupled with said outer cover and positioned to engage said elastic pad when said elastic pad is positioned between at least a portion of said outer cover and said elastic member to thereby hold said outer cover against said elastic pad.

3. The warming system of claim 1, wherein said first and second open ends define an air intake vent and a first air exhaust vent, respectively, said hollow air conveyor having a third open end that defines a second air exhaust vent such that said hollow air conveyor is shaped so as to define multiple air paths through said elastic pad that are respectively associated with different air exhaust vents.

4. The warming system of claim 1, wherein a plurality of different air chambers are defined within said hollow air conveyor such that a plurality of different air paths are defined through said elastic pad.

5. The warming system of claim 1, wherein said hollow air conveyor comprises a flame resistant material, a plurality of inner air vents being defined within said flame resistant material and respectively positioned adjacent to portions of said inner surface such that said portions of said inner surface are exposed to air that is located within said hollow air conveyor.

6. The warming system of claim 5, wherein said plurality of inner air vents are respectively positioned within said flame resistant material based upon a heat distribution associated with said elastic pad as a function of a preselected temperature range of said air.

7. The warming system of claim 5, wherein said first and second open ends define an air intake vent and an air exhaust vent, respectively, said air path beginning at said air intake vent and ending at said air exhaust vent, said plurality of inner air vents each having a different width or diameter, and said plurality of inner air vents being arranged within said flame resistant material such that an increasing air vent width or diameter sequence is defined along said air path.

8. The warming system of claim 7, wherein each of said different widths or diameters are selected based upon a heat distribution associated with said elastic pad as a function of a preselected temperature range of said air.

9. The warming system of claim 5, wherein said first and second open ends define an air intake vent and an air exhaust vent, respectively, said warming system further comprising:
a heat source configured to inject an amount of heated air into said air intake vent such that a first portion of said heated air escapes said hollow air conveyor through said plurality of inner air vents so as to warm said portions of said inner surface, and such that a second portion of said heated air escapes said hollow air conveyor through said air exhaust vent.

10. The warming system of claim 9, wherein first and second locking receptacles are associated with said heat source, said warming system further comprising:
a locking mechanism comprising:
a base; and
first and second locking extensions each configured to extend from said base to first or second extended positions, respectively, or retract toward said base to first and second retracted positions, respectively, said first and second locking extensions sized to engage said first and second locking receptacles, respectively, when said first and second locking extensions are in said first and second extended positions, respectively, so as to couple said heat source with said air intake vent such that said heat source is positioned to inject said heated air into said air intake vent.
